SEQUENCE LISTING (29 sequences)

SEQ ID NO: 1 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 3 - protein, 7 aa, Homo sapiens
CKVLRRH

SEQ ID NO: 4 - protein, 21 aa, Homo sapiens
HPLGSPGSASDLETSGLQEQR

SEQ ID NO: 5 - protein, 19 aa, Homo sapiens
LGSPGSASDLETSGLQEQR

SEQ ID NO: 6 - protein, 6 aa, Homo sapiens
NHLQGK

SEQ ID NO: 7 - protein, 25 aa, Homo sapiens
  misc_feature (25)..(25): Xaa can be any naturally occurring amino acid
LSELQVEQTSLEPLQESPRPTGVWX

SEQ ID NO: 8 - protein, 8 aa, Homo sapiens
EVATEGIR

SEQ ID NO: 9 - protein, 7 aa, Homo sapiens
MVLYTLR

SEQ ID NO: 10 - protein, 10 aa, Homo sapiens
MVQGSGCFGR

SEQ ID NO: 11 - protein, 9 aa, Homo sapiens
ISSSGLGCK

SEQ ID NO: 12 - protein, 4 aa, Homo sapiens
KMDR

SEQ ID NO: 13 - protein, 8 aa, Homo sapiens
  misc_feature (6)..(6): Xaa can be any naturally occurring amino acid
KMVLYXLR

SEQ ID NO: 14 - protein, 7 aa, Homo sapiens
  misc_feature (5)..(5): Xaa can be any naturally occurring amino acid
MVLYXLR

SEQ ID NO: 15 - protein, 8 aa, Homo sapiens
  misc_feature (4)..(4): Xaa can be any naturally occurring amino acid
EVAXEGIR

SEQ ID NO: 16 - protein, 10 aa, Homo sapiens
  misc_feature (1)..(1): Xaa can be any naturally occurring amino acid
XREVAXEGIR

SEQ ID NO: 17 - protein, 10 aa, Homo sapiens
  misc_feature (1)..(1): Xaa can be any naturally occurring amino acid
XREVATEGIR

SEQ ID NO: 18 - protein, 25 aa, Homo sapiens
  misc_feature (9)..(10): Xaa can be any naturally occurring amino acid
LSELQVEQXXLEPLQEXPRPXGVXK

SEQ ID NO: 19 - protein, 25 aa, Homo sapiens
  misc_feature (10)..(10): Xaa can be any naturally occurring amino acid
LSELQVEQTXLEPLQEXPRPXGVXK

SEQ ID NO: 20 - protein, 7 aa, Homo sapiens
LSELQVE

SEQ ID NO: 21 - protein, 18 aa, Homo sapiens
QTSLEPLQESPRPTGVWK

SEQ ID NO: 22 - protein, 17 aa, Homo sapiens
  misc_feature (3)..(3): Xaa can be any naturally occurring amino acid
QTXLEPLQEXPRPXXGV

SEQ ID NO: 23 - protein, 13 aa, Homo sapiens
LSELQVEQTSLEP

SEQ ID NO: 24 - protein, 12 aa, Homo sapiens
LQESPRPTGVWK

SEQ ID NO: 25 - protein, 12 aa, Homo sapiens
LSELQVEQTSLE

SEQ ID NO: 26 - protein, 13 aa, Homo sapiens
PLQESPRPTGVWK

SEQ ID NO: 27 - protein, 10 aa, Homo sapiens
SREVATEGIR

SEQ ID NO: 28 - protein, 8 aa, Homo sapiens
KMVLYTLR

SEQ ID NO: 29 - protein, 7 aa, Homo sapiens
MVLYTLR

(19) United States
(12) Patent Application Publication
(10) Pub. No.: US 2008/0312152 A1
(43) Pub. Date: Dec. 18, 2008

(54) Glycosylated Probnp

(76) Inventors: N. Stephen Pollitt, Los Altos, CA (US); Rodney A. Jue, Castro Valley, CA (US); Andrew A. Protter, Palo Alto, CA (US); Jessica O"Rear, Redwood City, CA (US); Andrew Guzzetta, Palo Alto, CA (US); Ute Schellenberger, Palo Alto, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/090,246

(22) PCT Filed: Oct. 16, 2006
(86) PCT No.: PCT/US2006/040436

§ 371 Date: Apr. 14, 2008

Related U.S. Application Data
(60) Provisional application No. 60/726,980, filed on Oct. 14, 2005.

(51) Int. Cl. A61K 38/00; C07K 14/00; G01N 33/00; A61P 43/00

(52) U.S. Cl. 514/12; 530/350; 436/86

(57) ABSTRACT

The present invention is directed to glycosylated proBNP and pharmaceutical compositions thereof. The present invention also relates to novel assays for measuring the total natriuretic activity that is present in a clinical blood sample.

### TECHNICAL FIELD

The present invention relates to glycosylated proBNP and pharmaceutical compositions thereof. It also provides for the use of glycosylated proBNP as a biomarker for related disease states.

### BACKGROUND ART

O-linked glycosylation has been found to occur on serum proteins and cell surface glycoproteins, as well as on larger hyperglycosylated secreted proteins called mucins Hounsell, E., Davies, M, and Renouf, D. (1996) Glycoconj J 13, 19-261). These carbohydrate moieties have diverse functions depending on the proteins to which they are attached. In the case of the mucins, which protect the lining of the respiratory and intestinal tracts, the massive degree of O-linked glycosylation is thought to maintain the polypeptide chain in an extended conformation thereby increasing the hydrodynamic radius of the protein seven fold over similarly sized globular domains Jentoft, N. (1990) Trends Biochem Sci 15, 291-4. This property is an important factor that accounts for the high viscosity of mucins. Cell surface glycoproteins have similar mucin-like domains that enable them to place ligand-binding regions of receptors at some distance from the cell surface. This spatial role has been determined to be critical for the function of the P-selectin/P-selectin glycoprotein 1 (PSGL-1) interaction which is responsible for the rolling action of neutrophils on activated endothelial cells Patel, K, Nollert, M, and McEver, R. (1995) J Cell Biol 131, 1893-902. O-linked carbohydrate has been found to modulate the stability, circulating half-life and activities of a number of serum glycoproteins including granulocyte colony stimulating factor (G-CSF), IgA1, and chorionic gonadotropin. See Oh-eda, M, Hasegawa, M, Hattori, K, Kuboniwa, H., Kojima, T., Orita, T, Tomonou, K, Yamazaki, T, and Ochi, N. (1990) J Biol Chem 265, 11432-5; Hasegawa, M. (1993) Biochem Biophys Acta 1203, 295-7; Jwase, H., Tanaka, A., Hiki, Y., Kokubo, T, Ishii-Karakasa, I., Kobayashi, Y., and Hotta, K (1996) J Biochem (Tokyo) 120, 92-7; Butnev, V., Gotschall, R., Baker, V, Moore, W., and Bousfield, G. (1996) Endocrinology 137, 2530-42. It has also been found to govern proteolytic processing of pro-opiomelanocortin. See Seger, M, and Bennett, H. (1986) J Steroid Biochem 25, 703-10.

Given the roles played by O-linked sugar and the increasing availability of sequence information from the several mammalian genomes, it would be beneficial to be able to make accurate predictions about the potential for O-linked glycosylation on unknown or poorly characterized proteins based solely on sequence data. Although numerous attempts have been made, no sequence motif has been found to control the addition of O-linked N-acetylgalactosamine (GalNAc) to serines and threonines in the same way that N-linked sugars are coupled to the Asn residue within the Asn-X-Ser/Thr motif. Nevertheless it has been noted that there appears to be a propensity for proline, serine and threonine as well as a negative influence of adjacent charged residues in the region surrounding the addition of carbohydrate. Studies have used the results of these surveys to deduce algorithms that will predict the sites of O-linked GalNAc addition with a reported accuracy of 70-90% (Hansen, J, Lund, O., Engelbrecht, J, Bohr, H., Nielsen, J., and Hansen, J. (1995) Biochem J 308 (Pt 3), 801-13). The accuracy of these prediction methods, however, depends on the data set on which they were developed and therefore the accuracy with which predictions about a newly discovered protein are made will depend on the degree to which that protein resembles proteins in the database.

Brain natriuretic peptide (BNP) is a member of the family of natriuretic peptides, which act on the cardiovascular system to reduce blood pressure and on the kidneys to increase sodium excretion (Nakao, K, Itoh, H., Saito, Y., Mukoyatna, M, and Ogawa, Y. (1996) Curr Opin Nephrol Hypertens 5, 4-11), (Ogawa, Y., Itoh, H., and Nakao, K (1995) Clin Exp Pharmacol Physiol 22, 49-53). Human BNP consists of a 32 amino acid peptide with a 17 amino acid disulfide loop structure. Human BNP is initially translated in the cell as a 134 amino acid protein containing a 26 amino acid signal peptide which presumably is rapidly removed during synthesis (Seilhamer et. al., Biochem Biophys Res Commun 165:650-658 (1989); Sudoh et al., Biochem Biophys Res Commun 159:1427-1434 (1989)). Once the signal peptide is removed a 108 amino acid BNP precursor protein, termed proBNP, is produced with the 32 amino acid BNP peptide located at the carboxyl-terminal end. The precursor has no N-link glycosylation motifs, and O-linked glycosylation is not predictable based on sequence data alone.

It is generally believed that the heart secretes a mixture of the proBNP protein as well as the mature BNP peptide into the blood. Levels of both forms become elevated in circulation in cases of congestive heart failure (Yandle, T G., Richards, A. M, Gilbert, A., Fisher, S., Holmes, S., and Espiner, E. A. (1993) J Clin Endocrinol Metab 76, 832-8), (Togashi, K., Fujita, S., and Kawakami, M. (1992) Clin Chem 38, 322-3) and correlate with the severity of heart failure. Hypertension and volume overload cause increased tension and stretching of the ventricular walls, and in response, proBNP is cleaved to BNP and N-terminal-proBNP. The role of N-terminal-proBNP is uncertain. BNP decreases blood pressure by vasodilation and renal excretion of sodium and water.

BNP exerts its biological effects by activating a specific cell surface receptor termed the guanylyl cyclase-A (GC-A) receptor or the NPR-A receptor. When activated, the receptor synthesizes cyclic GMP from GTP. Treatment of cells with BNP increases intracellular and extracellular concentrations of cyclic GMP. Furthermore, treatment of animals with BNP results in dose-dependent increases in cyclic GMP in the plasma. It is generally believed that the GC-A receptor and cyclic GMP mediates most if not all of the biological effects of BNP.

As an active hormone, BNP has a half-life of approximately 20 minutes. In plasma, BNP is inactived by two mechanisms, enzymatic hydrolysis and receptor-mediated endocytosis. Neutral endopeptidase, an endothelial cell-surface zinc metallo-enzyme, hydrolyzes the peptide. A natriuretic receptor, NPR-C, present in vascular wall, binds the peptide which is internalized by endocytosis and degraded. NPR-C has also a signalling function leading to vasodilation by activation of potassium channels.

### SUMMARY OF THE INVENTION

The present invention is based upon the discovery that endogenous proBNP is glycosylated, exhibits a longer plasma half-life, and has a lower activity than hBNP. Novel therapeutic compositions and novel assays are provided herein.

In one embodiment, the present invention is directed to glycosylated proBNP in an isolated and purified form. In a preferred embodiment, the present invention is directed to a pharmaceutical composition comprising glycosylated proBNP.

In another embodiment, the present invention is directed to assays that measure the total capacity of the blood to activate the natriuretic peptide pathway. In a preferred embodiment of the invention, the assay comprises the use of a soluble receptor of BNP as a reagent, preferably a soluble NPRA-Fc fusion protein that exhibits affinity for natriuretic peptides similar to the native receptor.

### BRIEF DESCRIPTION OF THE FIGURES

FIG. 1 is a gel analysis showing the deglycosylation of proBNP. Samples of CHO cell expressed proBNP were treated as indicated and analyzed by SDS-PAGE. Lane 1, untreated; Lane 2, N-acetylneuraminidase treated; lane 3, N-acetylneuraminidase and O-glycanase treated.

FIG. 2 is a tryptic peptide map of proBNP. ProBNP was digested with trypsin and separated by reverse phase capillary HPLC as described herein. Tryptic peptide designations are given above each peak with glycopeptides designated with a (g).

FIG. 3 provides source CID fragmentation of the T4+T5 peptide. LC/MS with source CID was conducted on a tryptic digest of asialo-proBNP. The data shown were collected from the region of the tryptic map corresponding to the absorbance peak shown in for the T4+T5 and T5 peptides. The inset shows the extracted ion current of the two peptides as a function of scan number within the single chromatographic peak. The mass spectrum was derived by averaging scans 708 to 711 (see inset). The [M+2H]2+ region of the spectrum is shown.

FIG. 4 is a schematic showing the proBNP sequence sites of carbohydrate addition. Glycosylated positions are indicated by open boxes if glycosylation is partial, filled boxes if complete. Tryptic peptide designations are given above the sequence and amino acid residue numbers beside the sequence. Portions of the protein not recovered and analyzed in the tryptic peptide map are shaded. Mature BNP consists of peptides T10 through T17.

FIG. 5 is a Western blot of pro-BNP in heart failure patient plasma demonstrating that natural human proBNP is glycosylated. The Triage® kit from Biosite was used to determine BNP levels.

FIG. 6 is a graph that demonstrates that the Triage® kit does not differentiate between hBNP and proBNP.

FIG. 7 is a graph showing competitive binding of glycosylated recombinant human proBNP to GC-A receptor relative to hBNP. ProBNP is less active in this receptor binding study than hBNP.

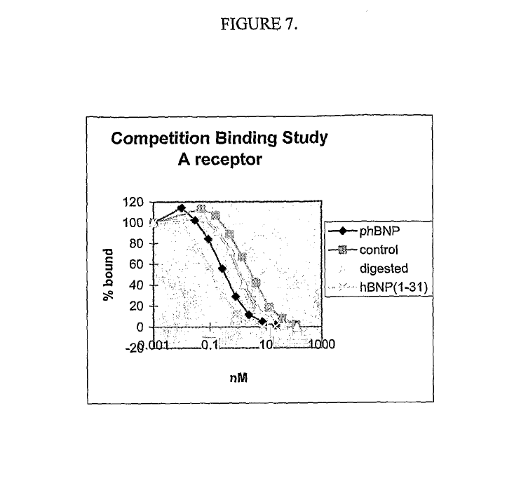

FIG. 8 is a graph showing the reduced potency of proBNP compared to hBNP on NPR-A activation in human aorta endothelial cells. The graph provides a direct activity comparison of hBNP relative to proBNP. As demonstrated in FIG. 15, NPR-A activation correlates to activation of the natriuretic mechanisms.

FIG. 9 is a graph providing the pharmacokinetic profiles in male Cyno monkeys of two i.v. doses of hBNP (1 and 3 nM/kg).

FIG. 10 is a graph providing the pharmacokinetic profile of an i.v. dose of proBNP in male Cyno monkeys (3 nM/kg).

FIG. 11 is a graph providing urinary cGMP levels in male Cyno monkeys after two i.v. bolus administrations of hBNP (1 and 3 nM/kg).

FIG. 12 is a graph providing urinary cGMP levels in male Cyno monkeys after an i.v. bolus administration of proBNP (1 nM/kg).

FIG. 13 is a graph providing urine output in male Cyno monkeys after two i.v. bolus administrations of hBNP (1 and 3 nM/kg).

FIG. 14 is a graph providing urine output in male Cyno monkeys after i.v. bolus administration of Pro-BNP (1 nM/kg).

FIG. 15. Demonstrates that NPR-A receptor activation correlates with inhibition of Ang II-induced Aldosterone secretion by human adrenal cortical cells.

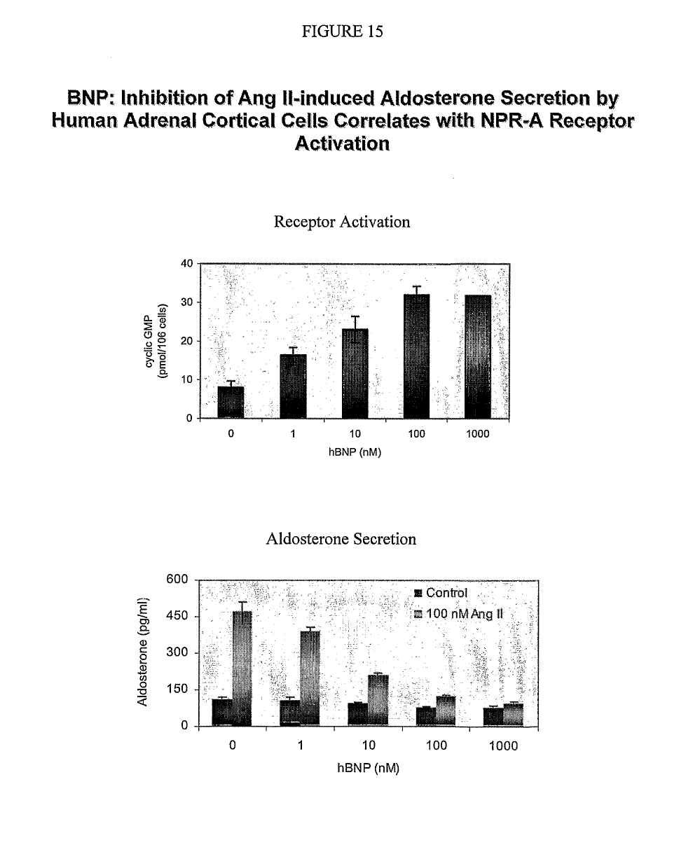

### DETAILED DESCRIPTION OF THE INVENTION

The present invention is directed to purified glycosylated proBNP, pharmaceutical compositions comprising said glycosylated proBNP, and their use for the treatment cardiac diseases such as congestive heart failure.

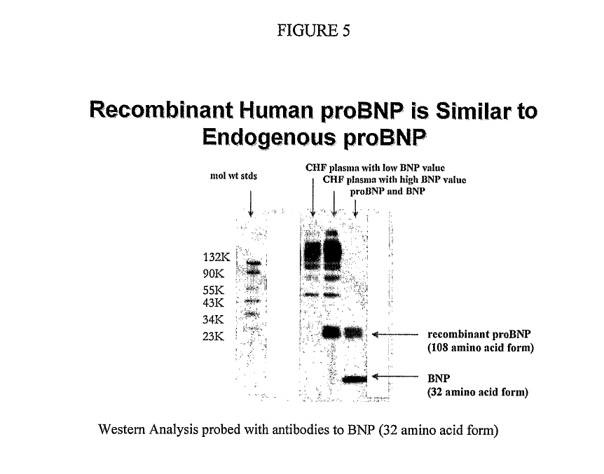

The present invention is based on the unexpected finding that both endogenous and recombinant human proBNP as expressed in Chinese Hamster Ovary (CHO) cells are glycosylated. Applicant has further discovered that said glysolyation is O-linked. The presence of at least seven points of carbohydrate addition within a 36 amino acid stretch of the propeptide constitutes a high concentration of glycosyl attachment and is unprecedented for a serum glycoprotein. As shown in FIG. 5, endogenous human proBNP is glycosylated. In isolated form, the O-link glycosylated human proBNP has pharmacokinetic profiles and biological effects which can be useful in pharmaceutical compositions and methods of treating congestive heart failure.

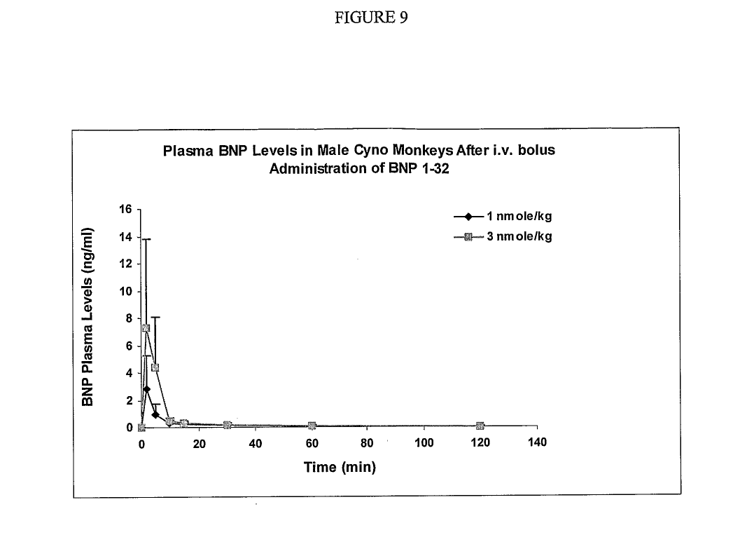

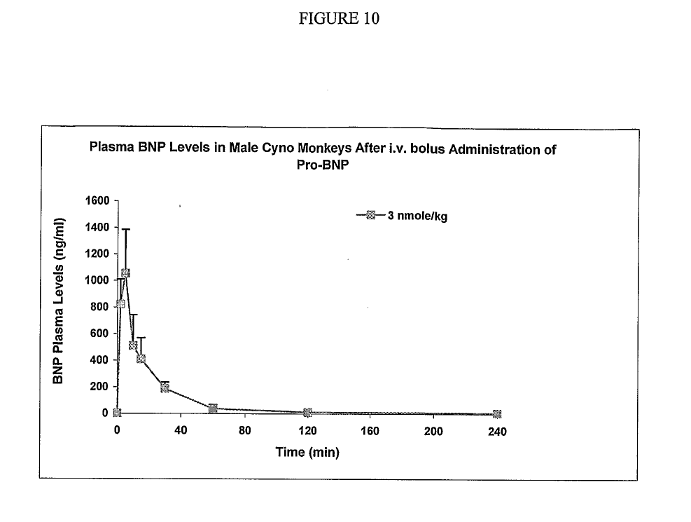

The present invention further provides that the glycosylated proBNP has a circulating half-life that greater than that of hBNP (See FIGS. 9 and 10). The prolonged circulating half-life is probably due to either a reduced rate of proteolytic degradation or a reduced rate of uptake by the clearance receptor.

With increased circulating half-life coupled with biological activities comparable to BNP, the glycosylated proBNP provides a useful therapeutic for treating heart diseases and heart failure. It can be even more desirable in treatments that prefer a longer circulating half-life of the substance, such as maintenance therapy after an acute heart failure.

Expression and Isolation of O-Link Glycosylated Human proBNP

Human proBNP can be expressed in eukaryotic cell lines, preferably mammalian cell lines, using recombinant techniques that are well known in the art. Transfected cells can be placed under drug selection so that a stable line expressing high levels of proBNP can be isolated. Levels of proBNP expression can be determined by a variety of protein detection methods, such as immunological methods using specific antibodies. Cell lines that stably express proBNP can be expanded and used to produce proBNP.

In a preferred embodiment, Chinese Hamster Ovary (CHO) cells are transfected with the gene encoding human preproBNP (SEQ ID:2), which is placed under the transcriptional control of the CMV promoter on a plasmid containing a glutamine synthase gene. Stable transfected cell lines can be generated by selection for resistance to methionine sulfoximine in glutaimne-free medium. Levels of human proBNP expression can be determined by ELISA. For production of proBNP, the cell line can be expanded to confluence with regular media changes.

A purified preparation of proBNP is contemplated as an embodiment of the presently disclosed invention. ProBNP can be purified using any methods known in the art. Preferably a proBNP-specific method of purification is used to purify human proBNP, for example, immunoaffinity chromatography. ProBNP-specific antibodies can be generated using a synthetic peptide harboring a stretch of proBNP sequence as immunogen, such as a peptide of proBNP coupled to BSA. An immunoaffinity column can be made using these antibodies. The immunoaffinity purified protein can be further purified by applying any other protein purification techniques, including, but not limited to, ion exchange chromotographies such as DEAE; size excusion chromatography; HPLC, such as reverse phase HPLC; and other methods that will be apparent to one skilled in the art upon reading the present disclosure.

### Structural Characterization of Recombinant ProBNP

Recombinant human proBNP can be characterized and glycosylation identified using a variety of methods that are well known in the art. The methods include, but not limited to, SDS-PAGE; amino acid analysis; Edman degradation; deglycosylation of the purified recombinant protein using enzymes that can remove carbohydrate moieties from protein, such as O-glycosidase or neuraminidase; proteolytic mapping with enzymes such as trypsin or Glu-C; mass spectrometry; and pulsed-liquid protein sequencing. For example, SDS-PAGE can be used to determine whether recombinant proBNP form a smear of multiple closely spaced bands, thus is likely glycosylated. Deglycosylation followed by mass spectrometry can confirm existent glycosylation on the protein. ProBNP fragments generated by proteolytic mapping can be separated by chromatography and subjected to mass spectrometry, which has the ability to detect glycosylated peptide and narrow the region where carbohydrates attach. To identify the exact glycosylation sites, Edman degradation and blank cycle sequencing can be used with purified proteolytic fragments.

Studies have demonstrated that hBNP induces a dose-related release of cyclic GMP from cells expressing the human guanylyl cyclase-A (GC-A), consistent with reports demonstrating that the GC-A receptor mediates most and probably all of the biological effects of hBNP and that cyclic GMP is an important second messenger for this receptor. Pursuant to the present invention, the effects of hBNP, unglycosylated proBNP, and O-link glycosylated proBNP on cyclic GMP release from cells expressing the human GC-A receptor were determined.

Previous studies using rabbits as an animal model have described pharmacokinetics and biological responses to hBNP including stimulation of plasma cyclic GMP, reducing blood pressure, diuresis, and natriuresis. Thus, in this study, the pharmacokinetics and biological effects of hBNP, unglycosylated proBNP, and glycosylated proBNP were determined and compared.

In vitro, using cells expressing the human GC-A receptor (also known as NPR-A receptor), O-link glycosylated proBNP was shown to be equivalent to hBNP in inducing cellular cyclic GMP release, a measure of receptor activation. O-link glycosylated proBNP was less potent than hBNP in this assay, indicating that it is a poorer ligand for hBNP's biological receptor.

In summary, glycosylated human proBNP has biological activities that are similar to human BNP. As demonstrated in FIGS. 9 and 10, proBNP exhibits a substantial increase in circulating half-life when compared to hBNP. These properties make proBNP an excellent therapeutic for use in conditions where exposure and rapid clearance are problems. Such conditions include chronic disorders or disease states including but not limited to congestive heart failure.

### Administration

Briefly, the glycosylated proBNP is useful in treatment of heart diseases and heart failure. The protein is administered in conventional formulations for peptides such as those described in Remington's Pharmaceutical Sciences, Mack Publishing Company, Easton, Pa. (latest edition). Preferably, the protein is administered by injection, preferably intravenously, using appropriate formulations for this route of administration. Dosage levels are on the order of 0.01-100 ug/kg of subject.

These compounds, and compositions containing them, can find use as therapeutic agents in the treatment of various edematous states such as, for example, congestive heart failure, nephrotic syndrome and hepatic cirrhosis, in addition to hypertension and renal failure due to ineffective renal perfusion or reduced glomerular filtration rate.

Thus the present invention also provides compositions containing an effective amount of compounds of the present invention, including the nontoxic addition salts, amides and esters thereof, which may, alone, serve to provide the above-recited therapeutic benefits. Such compositions can also be provided together with physiologically tolerable liquid, gel or solid diluents, adjuvants and excipients.

These compounds and compositions can be administered to mammals for veterinary use, such as with domestic animals, and clinical use in humans in a manner similar to other therapeutic agents. In general, the dosage required for therapeutic efficacy will range from about 0.001 to 100 ug/kg, more usually 0.01 to 100 ug/kg of the host body weight. Alternatively, dosages within these ranges can be administered by constant infusion over an extended period of time, usually exceeding 24 hours, until the desired therapeutic benefits have been obtained.

Typically, such compositions are prepared as injectables, either as liquid solutions or suspensions; solid forms suitable for solution in, or suspension in, liquid prior to injection may also be prepared. The preparation may also be emulsified. The active ingredient is often mixed with diluents or excipients, which are physiologically tolerable and compatible with the active ingredient. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

The compositions are conventionally administered parenterally, by injection, for example, either subcutaneously or intravenously. Additional formulations which are suitable for other modes of administration include suppositories, intranasal aerosols, and, in some cases, oral formulations. For suppositories, traditional binders and excipients may include, for example, polyalkylene glycols or triglycerides; such suppositories may be formed from mixtures containing the active ingredient in the range of 0.5% to 10% preferably 1%-2%. Oral formulations include such normally employed excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained-release formulations, or powders, and contain 10%-95% of active ingredient, preferably 25%-70%.

The protein compounds may be formulated into the compositions as neutral or salt forms. Pharmaceutically acceptable nontoxic salts include the acid addition salts (formed with the free amino groups) and which are formed with inorganic acids such as, for example, hydrochloric or phosphoric acids, or organic acids such as, for example, hydrochloric or phosphoric acids, or such organic acids as acetic, oxalic, tartaric, mandelic, and the like. Salts formed with the free carboxyl groups may be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In addition to the compounds of the present invention, which display natriuretic, diuretic or vasorelaxant activity, compounds of the present invention can also be administered through controlled release formulations or devices which are known to those skilled in the art. Such formulations and/or devices include albumin fusion peptides, transdermal delivery methods, and the like. Alternatively, by appropriate selection, compounds of the present invention whose activity levels are reduced or eliminated entirely can serve to modulate the activity of other diuretic, natriuretic or vasorelaxant compounds, including compounds outside the scope of the present invention, by, for example, binding to clearance receptors, stimulating receptor turnover, or providing alternate substrates for degradative enzyme or receptor activity and thus inhibiting these enzymes or receptors. When employed in this manner, such compounds can be delivered as admixtures with other active compounds or can be delivered separately, for example, in their own carriers.

Compounds of the present invention can also be used for preparing antisera for use in immunoassays employing labeled reagents, usually antibodies. Conveniently, the polypeptides can be conjugated to an antigenicity-conferring carrier, if necessary, by means of dialdehydes, carbodiimide or using commercially available linkers. These compounds and immunologic reagents may be labeled with a variety of labels such as chromophores, fluorophores such as, e.g., fluorescein or rhodamine, radioisotopes such as .sup.125 I, .sup.35 S, .sup.14 C, or .sup.3H, or magnetized particles, by means well known in the art.

These labeled compounds and reagents, or labeled reagents capable of recognizing and specifically binding to them, can find use as, e.g., diagnostic reagents. Samples derived from biological specimens can be assayed for the presence or amount of substances having a common antigenic determinant with compounds of the present invention. In addition, monoclonal antibodies can be prepared by methods known in the art, which antibodies can find therapeutic use, e.g., to neutralize overproduction of immunologically related compounds in vivo.

With respect to activating necessary or therapeutic natriuretic pathways in a patient in need thereof, proper assessment of a patient blood samples is critical. As provided herein, the present invention provides for a method to evaluate the capacity of a patient's blood to activate the natriuretic pathways. Understanding the concentrations and respective activities of hBNP and proBNP present in a blood sample is extremely useful for purposes of managing patient care. For example, a correct understanding of a patient's ability to activate the natriuretic pathway may lead the physician to cease, continue, increase, decrease, or otherwise modify treatment (e.g., increase the dosage of diuretic, ACE inhibitor, digoxin, O-blocker, calcium channel blocker, hBNP, and/or vasodialtor, or even consider surgical intervention).

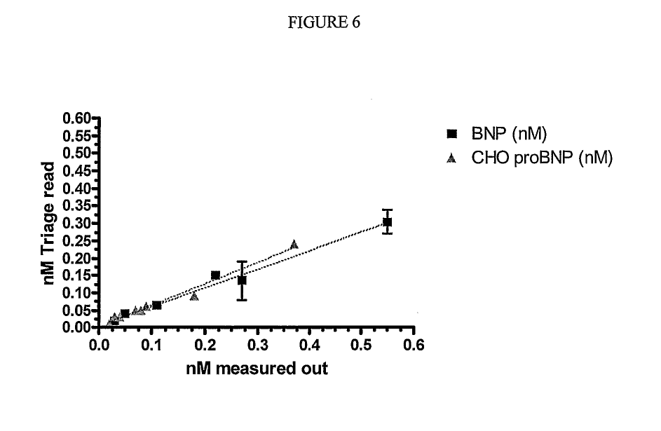

Understanding the respective activities of proBNP and hBNP in a clinical sample may also explain the so-called “endocrine paradox” in heart failure. As described by Goetze in Clin. Chem. 50: 1503-1510, 2004, heart failure patients have highly increased plasma concentrations of BNP. Surprisingly, however, these patients do not exhibit increased natriuresis. In fact, the opposite is true, as heart failure patients suffer from congestion, sodium retention, and edema. A further surprise is that these same patients do respond to administration of exogenous BNP with the expected increase in natriuresis. While not intending to be limited to a particular explanation for the endocrine paradox, it is likely that conventional assays used in the art do not monitor or take into consideration the ratio and respective activities of hBNP and proBNP in a patient sample. Most likely such assays do not differentiate between the different forms. See for example FIG. 6.

The following examples are offered to illustrate but not to limit the invention. All referenced cited herein are incorporated by reference in their entirety.

### EXAMPLE 1

### Recombinant Expression and Isolation of Human ProBNP

The gene encoding human preproBNP (SEQ ID:2) was placed under the transcriptional control of the CMV promoter on a plasmid containing a glutamine synthase gene. Chinese Hamster Ovary (CHO) cells were transfected by LIPOFECTAMINE (Gibco, Gaithersburg, Md.) as recommended by the manufacturer using 1 μg of plasmid DNA. Stable transfected cell lines were generated by selection for resistance to 10 μM methionine sulfoximine (MSX) (Davis, S. J., Ward, H. A., Puklavec, M. J, Willis, A. C., Williams, A. F., and Barclay, A. N. (1990) J. Biol Chem 265, 10410-815) in glutaimne-free GMEM-S (Bebbington, C., and Hentschel, C. (1987) in DNA Cloning (Glover, D., ed) Vol III, pp. 163-188, Academic Press, New York, J R H Bioscience, Lenexa, Kans.) with 10% dialysed fetal calf serum. Cells from this initial selection were pooled and replated in a 96 well plate at 5×10⁴ per well. The cells were subjected to selection for resistance to various levels of MSX from 100-700 μM. Levels of proBNP expression in each well were then determined by an ELISA. Cells from wells showing consistently high levels of production over the course of several media changes were subcultured and reassayed after growth to confluence. One cell line, 300-11D, was chosen for further work. For production of proBNP, the cell line was expanded to confluence in 1700 cm² roller bottles and media changes of 200 ml each were performed every three days.

A monoclonal antibody was developed using as an immunogen a synthetic peptide with the sequence, CKVLRRH, coupled via the cysteine sulfhydryl to BSA. The resulting mouse monoclonal, mAb8.1, requires the C-terminal His of BNP for binding. An immunoaffinity column was made by coupling the mAb8.1 antibody to UltraLink Hydrazide matrix (Pierce Chemical, Rockford, Ill.) according to manufacturer's directions. Binding capacity of a 10 ml column was 448 μg of synthetic BNP. The column was equilibrated in 0.1 M sodium phosphate buffer pH 7.1, and batches of 300-500 ml of conditioned media from the 300-11D transfected cell line were applied at a flow rate of 5 ml/min. The column was then washed in equilibration buffer and eluted with 0.1 M glycine pH 2.5. The eluted protein was collected based on monitoring absorbance at 280 nm. The immunoaffinity purified protein was applied to a 0.46×15 cm C4 reverse phase HPLC column (Vydac, Hesperia, Calif.) equilibrated in 10% acetonitrile, 0.1% TFA. The column was eluted by a gradient of 10-50% acetonitrile over 40 min. ProBNP elutes as a series of 2 or 3 unresolved peaks at about 23% acetonitrile which are well resolved from the elution time of mature BNP. The peaks do not differ in amino-terminal sequence and are apparently the result of glycosyl heterogeneity. The peaks were pooled and the protein was lyophilized.

### EXAMPLE 2

### Characterization of Recombinant Human ProBNP

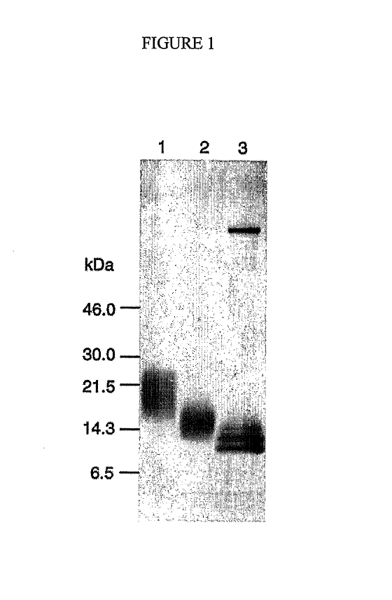

Automated pulsed-liquid Edman degradation of the purified protein gave two amino-terminal sequences: one derived from the known amino-terminus of proBNP as determined by Hino et al. (Hino, J., Tateyama, H., N., M, Kangawa, K, and Matsuo, H. (1990) Biochem Biophys Res Comm 167, 693-700) and a second sequence of roughly equal abundance lacking the amino-terminal His-Pro dipeptide. SDS-PAGE of purified recombinant proBNP (FIG. 1) gave rise to a smear of multiple closely spaced bands centered around 20 KDa.

Deglycosylation reactions were carried out in 250 mM sodium phosphate buffer, pH 6.0 at 37° C. with O-glycosidase or N-acetylneuraminidase (NANaseIII), both obtained from Glyko (Novato, Calif.). Digestion of the protein with N-acetylneuraminidase caused a reduction in the size of the smear as well as the apparent average mass of protein to approximately 18 KDa. Since there are no sites for N-link glycosylation, further digestion of the neuraminidase-treated material was carried out with O-glycosidase. This resulted in a predominant band at about 12 KDa and a secondary band at 14 KDa which is apparently due to incomplete deglycosylation.

To further characterize the recombinant protein, electrospray MS of the deglycosylated preparation was performed on a Finnigan SSQ 7000 mass spectrometer (San Jose, Calif.) in the positive ion mode. All LC/MS was performed using a capillary reverse phase column with a flow rate into the mass spectrometer of 5 μL/min. Nebulization was assisted with an auxiliary 5 μL/min flow of 2-methoxy ethanol. The mass spectrometer was scanned from m/z 300 to 2000 with a scan duration of 3 sec. Source collision induced dissociation (CID) was performed with an octapole offset of 30 v.

Electrospray MS of the deglycosylated preparation gave a predominant peak in the deconvoluted spectrum of 11,902.2 dal with a secondary peak at 11,669.3 dal corresponding to loss of the amino-terminal His-Pro dipeptide. Forms corresponding to the 14 KDa SDS-PAGE band were not detected, possibly due to lack of abundance and mass heterogeniety.

### EXAMPLE 3

### Determination of Glycosidic Addition Sites and Carbonhydrate Composition

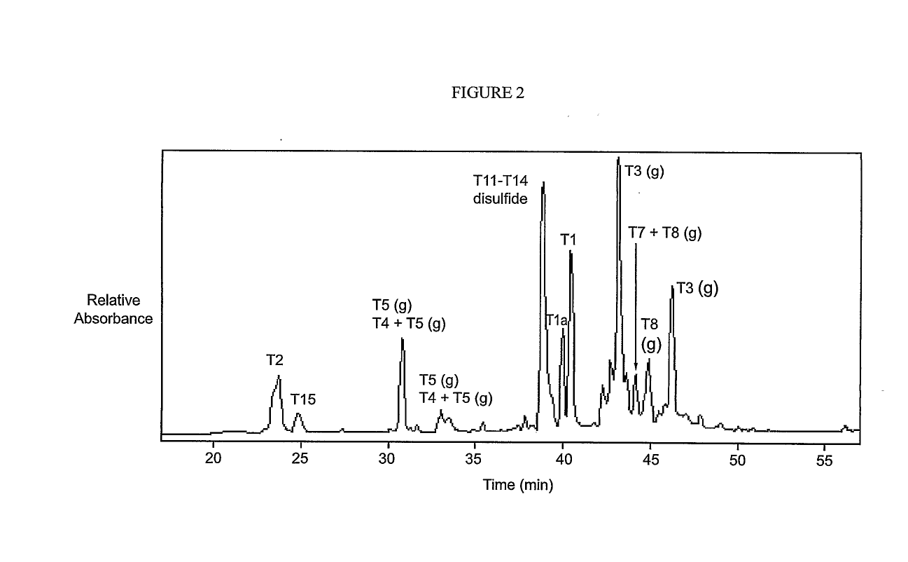

To determine glycosidic attachment sites, the recombinant proBNP was subjected to tryptic mapping. 127 μg of proBNP was first deglycosylated by digestion with either neuraminidase and O-glycosidase or neuraminidase alone in 250 mM sodium phosphate buffer, pH 6.0 at 37° C. Concentrated buffer was added to achieve a final concentration of 50 mM TrisHCl, pH 8.0, and 1 μg trypsin was added. Digestion was allowed to proceed overnight at room temperature. The digested protein was subjected to LC/MS (see FIG. 2, and Table 1). Peptide maps were generated using capillary HPLC as follows: Capillary flow (5 μL per min) was established by split flow from an HP 1090 HPLC PV5 (Hewlett-Packard, Palo Alto, Calif.) run at a flow rate of 200 μL per min. Chromatography was performed on a VYDAC C 18 0.32×250 mm column (Microtech Inc., Sunnyvale, Calif.) maintained at 40° C. Asialo-proBNP (30 μmol) was injected onto the column after equilibration with 0.1% TFA. The tryptic fragments were eluted with a gradient to 30% acetonitrile over 40 min and were collected for N-terminal peptide sequencing.

The non-glycosylated peptides were identified from the LC/MS map by mass and then confirmed in a subsequent LC/MS run using source CID to fragment the peptides. The glycosylated peptides were identified through the characteristic carbohydrate marker ions (oxonium ions) using a method described by Carr et al. (18).

TABLE 1
Masses and Amino Acid Sequences Determined for Neuraminidase Treated Tryptic Peptides

| Tryptic | Retention | Residue | Expected | Observed | | |
|---|---|---|---|---|---|---|
| Peptide | Time (min) | Number | Mass | Mass | Δ Mass | Structure |
| T1 | 40.4 | 1-21 | 2166.3 | 2166.2 | −0.10 | HPLGSPGSASDLETSGLQEQR |
| T1a | 39.9 | 3-21 | 1932.0 | 1931.5 | −0.50 | LGSPGSASDLETSGLQEQR |
| T2 | 23.6 | 22-27 | 695.8 | 695.5 | −0.29 | NHLQGK |
| T3^{a,d} | — | 28-52 | ^{c} | ^{c} | — | LSELQVEQTSLEPLQESPRPTGVWK |
| T4 | — | 53-54 | — | — | — | SR |
| T5 | 33.0 | 55-62 | 874.0 | 874.0 | −0.03 | EVATEGIR |
| T6 | 8.3 | 63-65 | 368.2 | 368.2 | 0.00 | GHR |
| T7 | — | 66 | — | — | — | K |
| T8^{a,d} | — | 67-73 | ^{c} | ^{c} | — | MVLYTLR |
| T9 | 11.0 | 74-76 | 342.4 | 342.3 | −0.10 | APR |
| T10 | 10.4 | 77-79 | 330.4 | 330.1 | −0.3 | SPK |
| T11-T14^{b} | 38.8 | (80-89)- | 1977.3 | 1977.0 | −0.29 | MVQGSGCFGR |
| | | (94-103) | | | | ISSSGLGCK |
| T12 | — | 90 | 146.2 | — | — | K |
| T13 | — | 91-93 | 420.5 | — | — | MDR |
| T12 + T13 | 12.3 | 90-93 | 548.7 | 548.3 | −0.36 | KMDR |
| T15 | — | 104-106 | 386.5 | — | — | VLR |
| T16 | — | 107 | 174.2 | — | — | R |
| T17 | — | 108 | 155.2 | — | — | H |

^{a}Glycosylated peptides.
^{b}Peptides are disulfide linked.
^{c}Results are presented in Table 2.

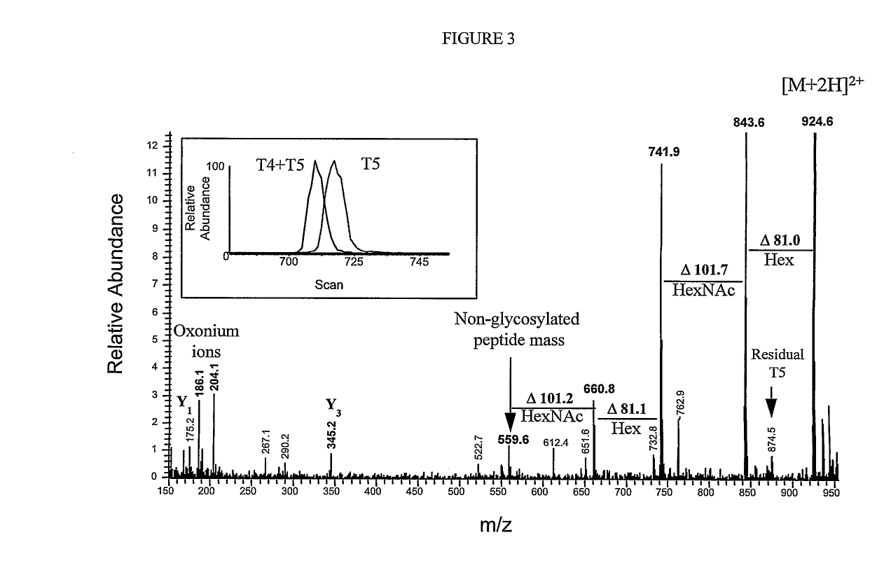

During source CID the carbohydrate moiety absorbs most of the collisional energy and fragments while the peptide portion of the glycosylated peptide remains intact. In all cases source CID was capable of striping off all of the carbohydrate to reveal the mass of the expected peptide. As an example, the CID mass spectra of the T4+T5 glycopeptide is shown in FIG. 3. This peptide appears to elute in a single peak with the T5 peptide, however extracted ion plotting of the two peptides reveals that the more heavily glycosylated T4+T5 peptide elutes slightly earlier as expected (see FIG. 3 inset). Clearly shown at the low mass end of the spectrum are the oxonium ions at m/z=204 and 186, derived from HexNAc and HexNAc—H₂O respectively. These ions indicate the presence of a glycosylated peptide. Also noted are minor ions at m/z=175.2 and 345.2 which correspond to the Y1 and Y3 ions respectively. The doubly charged ion of the fully glycosylated parent mass 1848.9 is noted at m/z=924.6. Differences of HexNAc and hexose monosaccharide units are noted as doubly charged mass differences from the parent mass. The sugars are stripped off down to the fully unglycosylated doubly charged peptide at m/z=559.5. To confirm the site of carbohydrate attachment the peptides were collected after capillary reverse phase HPLC and submitted for Edman degradation. The sites of attachment could then be determined through blank cycle sequencing (Pisano, A., Redmond, J. W., Williams, K. L., and Gooley, A. A. (1993) Glycobiology 3, 429-35).

For sequencing analysis, isolated proBNP tryptic peptides (10-20 picomoles) were spotted on BIOBRENE pre-cycled glass fiber filters and sequenced on an APPLIED BIOSYSTEMS 494 PROCISE PROTEIN SEQUENCER (Perkin Elmer, Applied Biosystems Division; Foster City, Calif.) using the pulsed-liquid reaction cycle. PTH amino acids were separated on an APPLIED BIOSYSTEMS 140C PTH ANALYZER. ProBNP (200 picomoles) was spotted on BIOBRENE precycled glass fiber filter and sequenced on an APPLIED BIOSYSTEMS 477A PROTEIN SEQUENCER using the Normal-1 reaction cycle. PTH amino acids were separated on an APPLIED BIOSYSTEMS120A PTH ANALYZER. All sequencing reagents and solvents were purchased from the instrument manufacturer.

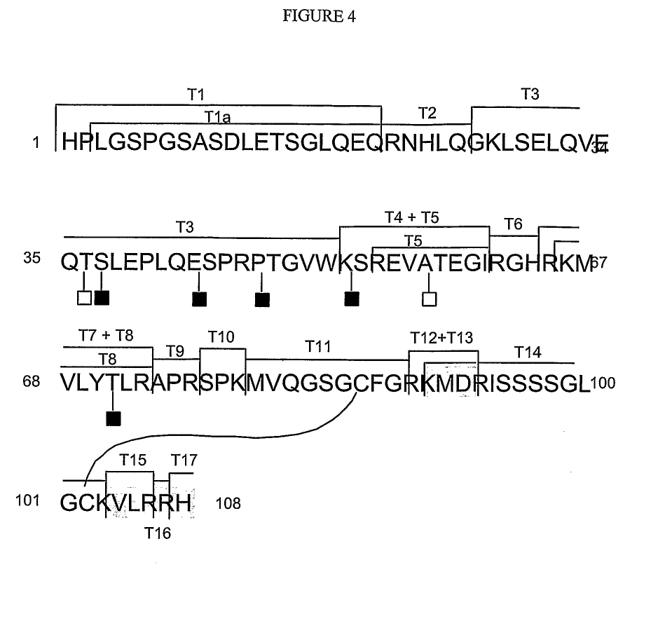

Sequence analysis of peaks at 44.2 and 44.9 min in the tryptic map (FIG. 2) yielded sequences of KMVLYXLR and MVLYXLR, respectively, which correspond to T7+T8 and T8 (FIG. 4). The absence of a detectable threonine at position 6 in the 44.2 min peak and position 5 in 44.9 min peak confirms that Thr-71 of SEQ ID: 1 is glycosylated.

Mass determination of peaks on the tryptic map at 30.8 min and 33.0 min identified that both of these peaks consisted of mixtures of T4+T5 and T5. Edman degradation of the peak at 30.8 min yielded two sequences, EVAXEGIR and XREVAXEGIR, indicating glycosylation of Ser-53 and Thr-58 of SEQ ID1. The peak at 33.0 min also yielded mixed sequences of EVATEGIR and XREVATEGIR, again indicating glycosylation of Ser-53 but unlike the 30.8 min fraction giving good recovery of Thr on cycle 4. This indicates that glycosylation of Thr-58 is partial. It is important to note that the T4 dipeptide was not isolated except as part of the T4+T5 peptide. It is possible that Ser-53 is also partially glycosylated and that this feature determines the ability of trypsin to cleave after Aig-54.

Sequence analysis of fractions with retention times of 43.2 min and 46.3 min from the tryptic map (FIG. 2) yielded sequences of LSELQVEQXXLEPLQEXPRPXGVXK and LSELQVEQTXLEPLQEXPRPXGVX(K), respectively corresponding to tryptic peptide T3. The absence of detectable serine at position 10 in both peptides implicates Ser-37 as the site of glycosyl attachment while the recovery of serine at position 2 in both peptides shows that Ser-29 is not glycosylated. Glycosylation of Thr-36 is partial and the presence of the glycosyl moiety in the 43.2 min peak appears to be the basis for separation of the two peptides. No signal is seen at positions 17 and 21 in either peptide, indicating that Ser-44 and Thr-48 may also be glycosylated but the lack of signal may also be due to low recovery of serine and threonine which can happen farther into the sequencing regime.

Amino acid sequencing of the more hydrophobic T3 peptide gave blank cycles for positions 9, 10, 17, and 21 implicating residues Thr-36, Ser-37, Ser-44, and Thr-48 as points of glycosyl attachment. Sequencing of a larger amount of peptide (200 μmol) strengthened assignment of the later cycles. LC/MS revealed that the peptide was selectively cleaved after Glu-34 to give the following peptides LSELQVE and QTSLEPLQESPRPTGVWK. These experiments also showed the LSELQVE-containing peak to be unglycosylated while the QTSLEPLQESPRPTGVWK-containing peak showed a mass consistent with a (HexNAc-Hex)₃ glycosyl structure. Sequencing of the two peptides gave amino terminal sequences LSELQVE and QTXLEPLQEXPRPXXGV with blank cycles corresponding to residues Ser-37, Ser-44, and Thr-48 once again implicating these as the sites of glycosyl attachment. This result supports the previous sequencing of the T3 tryptic peptides.

Table 2 shows the deduced carbohydrate composition based on the observed mass of each of the glycopeptides in the tryptic digest. For the simple glycopeptides having one or two attachment sites, mass correlation to the proposed structure was within 0.6 dalton. For the more complex structures obtained from peptide T3, observed masses occasionally gave discrepancies as great as 3.1 dalton. Mass accuracy for these species is reduced owing to lower abundance of the individual species giving rise to lower spectral intensities. Comparison of carbohydrate composition to the number of attachment sites shows that most sites appear to have a single Hex-HexNAc, most likely similar to the type 1 core sequence, Gal□1-3GalNAc (1). Peptide T3 shows a complex and heterogeneous glycosylation pattern characterized by a number of species having an unbalanced number of Hexose and HexNAc residues as has been previously observed in many branched chain structures in CHO cells (Dennis, J (1993) Glycobiology 3, 91-96). The pattern of glycosylation on the T3 tryptic peptide eluting at 43.2 is almost precisely repeated on the T3 peptide having an extra glycosylation site at Thr-36 (46.3 min elution time) with the exception of the addition of an extra HexNAc-Hex subunit to each glycoform.

TABLE 2
Predicted Glycosyl Composition based on Mass Spectral Data

| | | | Peptide | | | | |
|---|---|---|---|---|---|---|---|
| Tryptic | Retention | | Residue | Carbohydrate | Observed | Expected | |
| Peptide | time (min) | Peptide Sequence^{a} | Number | Composition | Mass | Mass | Δ Mass |
| T3 | 43.2 | LSELQVEQTSLEP | 28-52 | (HexNAc + Hex) + HexNAc | 3420.7 | 3419.6 | 1.1 |
| | | LQESPRPTGVWK | 28-52 | HexNAc₃ | 3461.8 | 3461.6 | 0.2 |
| | | | 28-52 | (HexNAc + Hex)₂ | 3582.9 | 3582.0 | 0.9 |
| | | | 28-52 | (HexNAc + Hex) + HexNAc₂ | 3623.9 | 3622.4 | 1.5 |
| | | | 28-52 | (HexNAc + Hex)₂ + HexNAc | 3786.1 | 3785.0 | 1.1 |
| | | | 28-52 | (HexNAc + Hex)₃ | 3948.2 | 3946.8 | 1.4 |
| | | | 28-52 | (HexNAc + Hex)₂ + HexNAc₂ | 3989.3 | 3992.4 | −3.1 |
| | | | 28-52 | (HexNAc + Hex)₃ + HexNAc | 4151.2 | 4152.3 | −1.1 |
| | | | 28-52 | (HexNAc + Hex)₄ | 4313.6 | 4314.8 | −1.2 |
| | | | 28-52 | (HexNAc + Hex)₄ + HexNAc + dHex | 4662.9 | 4661.4 | 1.5 |
| T3 | 46.3 | LSELQVEQTSLE | 28-52 | HexNAc | 3055.4 | 3054.4 | 1.0 |
| | | PLQESPRPTGVWK | 28-52 | HexNAc + Hex | 3217.5 | 3216.8 | 0.7 |
| | | | 28-52 | (HexNAc + Hex) + HexNAc | 3420.7 | 3419.8 | 0.9 |
| | | | 28-52 | (HexNAc + Hex)₂ | 3582.9 | 3581.8 | 1.1 |
| | | | 28-52 | (HexNAc + Hex) + HexNAc₂ | 3623.9 | 3621.8 | 2.1 |
| | | | 28-52 | (HexNAc + Hex)₂ + HexNAc | 3786.1 | 3785.8 | 0.3 |
| | | | 28-52 | (HexNAc + Hex)₃ | 3948.2 | 3946.8 | 1.4 |
| | | | 28-52 | (HexNAc + Hex)₃ + HexNAc + dHex | 4297.6 | 4296.9 | 0.7 |
| T4 + T5 | 30.8 | SREVATEGIR | 53-62 | (HexNAc + Hex)₂ | 1847.3 | 1847.9 | −0.6 |
| T4 + T5 | 33.0 | SREVATEGIR | 53-62 | (HexNAc + Hex) | 1482.6 | 1482.6 | 0.0 |
| T5 | 30.8 | EVATEGIR | 55-62 | (HexNAc + Hex) | 1239.2 | 1239.3 | −0.1 |
| T7 + T8 | 44.2 | KMVLYTLR | 66-73 | (HexNAc + Hex) | 1388.1 | 1388.6 | −0.5 |
| T8 | 44.9 | MVLYTLR | 67-73 | (HexNAc + Hex) | 1260.0 | 1260.5 | −0.5 |

^{a}Underlined amino acid residues are glycosyl attachment points based on blank cycle sequencing.

### EXAMPLE 4

### Natural Human ProBNP is Glycosylated

A Western Analysis of blood samples from congestive heart failure (CHF) patients, recombinant (CHO) produced proBNP, and HBNP was conducted. Antibodies to human BNP (1-32) were used to immunoprecipitate BNP cross-reacting material from human plasma, which was then subjected to Western blot analysis along with CHO-produced proBNP. The results depicted in FIG. 5 show that the immunoprecipitates from human plasma containing high levels of BNP as determined by Biosite Triage® BNP assay kit, gave rise to a band comigrating with CHO cell derived proBNP. This band was absent in immunoprecipitates from human plasma containing low levels of BNP. Bands of higher molecular weight present in Western lanes from both human plasma immunoprecipiates are due to IgG. FIG. 6 shows that both CHO expressed proBNP and BNP (1-32) react equally in the Triage® test.

### EXAMPLE 5

### Potency of Glycosylated proBNP and hBNP with Respect to NPR-A Activation in Human Aorta Endothelial Cells

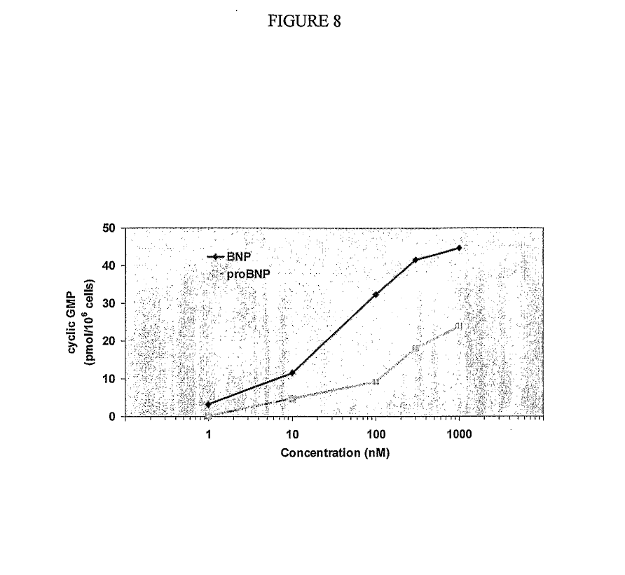

As shown in FIG. 8, both proBNP and hBNP exhibit activity against the NPR-A receptor. With respect to proBNP and BNP, total “Natriuretic Activity” in a blood sample is defined by the cumulative activity of proBNP and hBNP. When measuring against this receptor however it should be appreciated that the activity of other relevant natriuretic peptides, such as ANP and proANP can and should also be taken into consideration. The present invention provides for any sequence variations (as to length, amino acid substitutions, deletions, and the like) that are substantially similar to proBNP and/or BNP as long as they demonstrate activity against the NPRA-receptor and are glycosylated.

### EXAMPLE 6

### Comparative Pharmacokinetic and Renal Effects of BNP and proBNP in Cynomolgus Monkeys

Procedure for Intravenous Bolus Injection of BNP and proBNP:

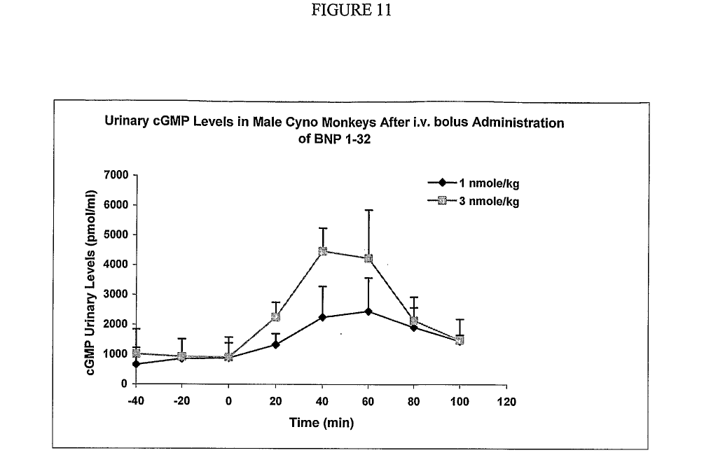

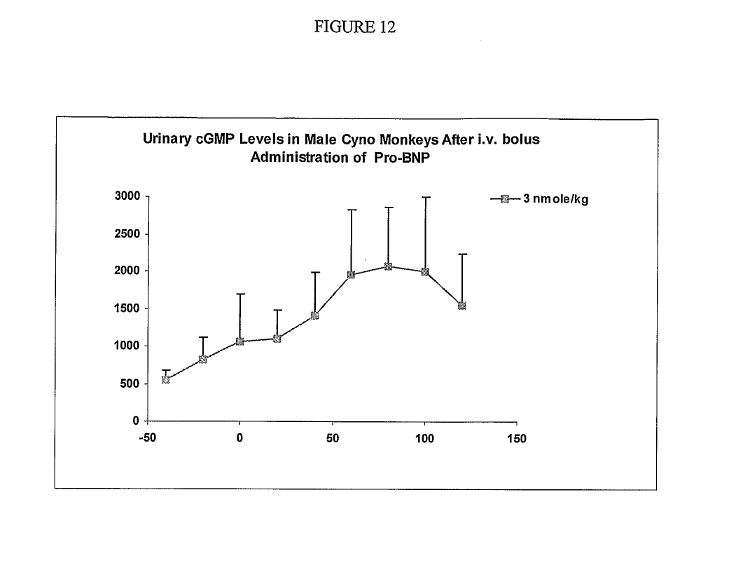

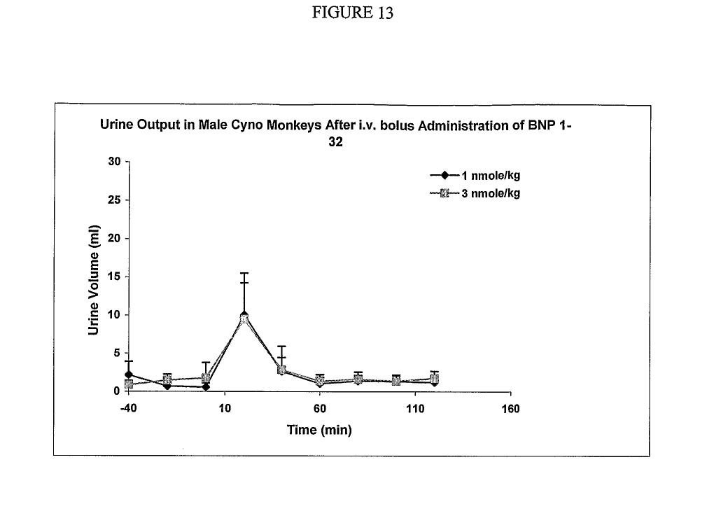

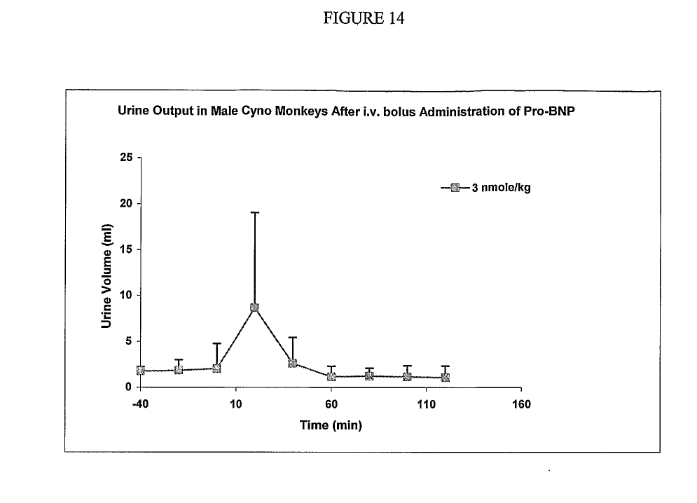

Six adult male cynomolgus monkeys (5-6 kg) from the same colony were randomly selected for this study. In the morning of the experiment day, each monkey was quickly anesthetized by inhalation of 5% isoflurance/95% oxygen. Once the animal was unconscious, the isoflurance was reduced to 1.5% and a sterile catheter was inserted into the urinary bladder. Another catheter was connected to a cephalic vein in the right or left arm of the monkey for compound delivery. The anesthesia was discontinued. The conscious monkey was seated in a restraining chair and allowed to stabilize for 1 hour. The conscious monkey received two bolus doses (1 nmol/kg and 3 nmol/kg) of each human BNP analog in 1 ml of saline via cephalic vein injection followed by a flush with 3 ml of saline. One hour of washing-out period was required between the two administrations. Two ml of blood were drawn into a EDTA tube containing 150 kallikrein-inactivating units aprotonin via a cephalic vein in the another arm of the monkey at the following 8 time points: baseline (within 2 min prior to dosing), 2, 5, 10, 15, 30, 60 and 120 min. The collected samples were kept on ice prior to centrifugation at 4° C. The plasma from each time point was aliquoted to 4 Eppendorff tubes with approximately 250 ml/tube. For urine collection, the bladder was emptied and flushed with 5 ml of sterile water. The urine was collected to a 15 ml regular polypropylene tube in every 20 min at the following time points: −60, −40, −20, 0, 20, 40, 60, 80, 100 and 120 min. Weighing of tube was required before and after collection. The urine sample from each time point was aliquoted to 4 Eppendorf tubes with 250 ml/tube. All plasma and urine samples were kept at −80° C. and delivered on dry ice. Material supply: 166 mg and 499 mg of hBNP were required for 8 cynos with the body weight of 6 kg at the doses of 1 nmol/kg and 3 nmol/kg, respectively. The material for each dose was weighed to a 15 ml sterile tube and dissolved in 8 ml of sterile saline prior to injection. Approximately 1 ml of the material at each dose was injected to each animal. The injection volume (ml) of the material to each animal was equal to animal body weight (kg) divided by six. Results comparing BNP to proBNP are presented in FIGS. 8 through 14. In summary, in conscious restrained cynomolgus monkeys, proBNP presented an extended PK profile compared to BNP. Secondly, when compared to BNP, proBNP demonstrated reduced effects relative to cGMP levels in both plasma and urine. Interestingly BNP and proBNP had similar effects on urine output. In conclusion, the data shows that proBNP is not metabolized in the blood.

All references provided herein are hereby incorporated by reference in their entirety.

1. A purified polypeptide comprising the amino acid sequence of SEQ ID:1 wherein one or more amino acids of said polypeptide is glycosylated.

2. The polypeptide of claim 1 comprising glycosylated serine.

3. The polypeptide of claim 1 comprising glycosylated threonine.

4. (canceled)

5. The polypeptide of claim 1 wherein one or more of the glycosylated amino acids is selected from the group consisting of Thr-36, Ser-37, Ser-44, Thr-48, Ser-53, Thr-58, Thr-71.

6. A pharmaceutical composition comprising the polypeptide of claim 1 or a pharmaceutically acceptable salt thereof.

7. The composition of claim 1 comprising a therapeutically effective amount of said polypeptide in admixture with a pharmaceutically acceptable carrier.

8. A method for the treatment of a cardiac, renal or inflammatory disease, comprising administering a therapeutically effective amount of the polypeptide of claim 1 to a patient in need thereof.

9. A method for measuring the total natriuretic activity in a blood sample, said method comprising identifying relative amounts of proBNP and BNP that are present in said sample.

10. The method of claim 9 comprising the use of a soluble NPRA-SC fusion protein.

11. The method of claim 8 wherein said disease is heart failure.

12. The method of claim 11 wherein said disease is chronic heart failure.
